(19) United States
(12) Patent Application Publication
(10) Pub. No.: US 2005/0148998 A1
(43) Pub. Date: Jul. 7, 2005

(54) Tactile applicator of liquids in packets

(76) Inventor: Jeffrey T. Haley, (US)

Correspondence Address: GRAYBEAL, JACKSON, HALEY LLP, 155 - 108TH AVENUE NE, SUITE 350, BELLEVUE, WA, 98004-5901, US

(21) Appl. No.: 10/749,633

(22) Filed: Dec. 31, 2003

(51) Int. Cl. A61M 31/00

(52) U.S. Cl. 604/520; 604/218

(57) ABSTRACT

A toothbrush-shaped self-dosing applicator for applying drops of liquid to specific locations in the mouth. The applicator includes a tactile surface at the tip which a user can sense to guide placement of the applicator by feel. A hole in the tip of the applicator under the tactile surface or beside the tactile surface or opposite the tactile surface releases a liquid onto the intended spot in the mouth. The liquid may be retained in a packet inside the tip of the applicator until a plunger is pressed, rupturing the packet.

### BACKGROUND

For topical treatment of health problems in the mouth, there are numerous liquids, gels, or pastes, all of which are hereinafter referred to as “liquid”, that are applied to a spot in the mouth. When applied by a mouth care professional, the professional has adequate vision, sometimes aided by a small mirror on a handle, for guiding placement of the liquid. However, when a person with a mouth sore attempts to self-apply such a product without the aid of another person who can see the sore, existing applicators are inadequate for several reasons.

First, because of the fixed relation between the eyes and mouth, a person cannot get multiple views into their own mouth. From the single available view, many mouth sores are difficult to see. Second, when an applicator is placed in the mouth, the applicator or the hand holding it often blocks the view of the sore from this single perspective. Third, limitations of this perspective make it difficult to judge the distance that the applicator is extending into the mouth, so it is difficult to judge when the tip of the applicator is directly over the sore. In most cases, the person wishes to place the treatment liquid only on the sore and not other places, and this depth perception problem causes unintentional placement of the treatment liquid in front of or behind the sore in the perception dimension.

Existing applicators for placing a drop of liquid in the mouth will apply the liquid to the first surface they touch. When a person is applying the liquid in their own mouth, they often mistakenly touch an undesired spot before they touch the preferred spot. An applicator is needed that places no liquid on a spot touched by the applicator until the applicator is properly placed and manually activated. It would be helpful for spreading the liquid if the applicator includes a generally low curvature surface surrounding the hole where the liquid comes out so that the surface will help spread the liquid as it emerges.

### SUMMARY OF THE INVENTION

In one aspect, the invention is a tactile applicator for placing a dose of liquid in a preferred spot in the mouth using the sense of touch to guide the applicator rather than the sense of vision. People have a very acute sense of touch in the tips of their tongues. A person can feel with their tongue the location of a sore and the location of the tip of an applicator. In this aspect, the applicator is a long, thin device like a toothbrush without bristles. Instead of bristles, the applicator has a hole on one side of the tip from which the treatment liquid is expressed at the moment desired by the person. Several means for causing the liquid to exit the hole at a desired moment are possible. In one embodiment, the device is a sheath with a plunger. The person activates the plunger to express the liquid out the hole in the tip of the sheath.

In one embodiment of the invention, at the tip of the sheath, there is a tactile surface which can be felt by the tip of the tongue or other tissues in the mouth and can be easily distinguished from other parts of the sheath. The tactile surface may be just beside the hole or surround the hole or, if it is porous so the liquid can flow through it, cover the hole. Or it may be on the opposite side of the tip of the sheath from the hole. In this embodiment, because the tactile surface is placed opposite the hole, it can be used to set the tactile surface directly over the sore using sensitivity of the sore as a guide. Then, the applicator can be rotated 180 degrees to place the hole directly over the sore. To facilitate accuracy of the 180-degree rotation of the applicator, the exterior surface of the applicator is preferably non-round in shape. The non-round shape is preferably symmetrical about a plane passing through a longitudinal axis of the applicator, such as ovoid (encompassing elliptical and straight sided ovals). When the exterior of the applicator is ovoid in shape, it is easy to feel with both the tissues of the mouth and with the fingers when the applicator has been rotated 180-degrees.

For many mouth sore treatment liquids to be applied, it is preferable to remove saliva and any mucous from the sore before the liquid is applied. For this purpose, in one embodiment, the tactile surface is an absorbent pad such as made of cotton or other absorbent fiber. The pad has a distinctly different feel from the smooth surface of the sheath and is easy to locate with the tongue or other touch sensitive tissues. If the sore is particularly sensitive, it is easy to locate the absorbent pad directly on the sore where it then absorbs saliva and mucous from the sore before the liquid is expressed out of the tip of the applicator. If the hole is opposite the absorbent pad, the user rotates the applicator 180 degrees and presses the plunger to express the liquid onto the sore immediately after it has been cleared of saliva and mucous.

In one embodiment, the applicator is a sheath and a plunger forming a syringe. The plunger may include a rubber head, like a typical disposable syringe, and the syringe may be filled in typical fashion by suction just before applying the liquid in the mouth. However, it is preferable to fill the syringe before it is dispensed to the consumer. This requires that the hole be blocked until the moment of application. For this purpose, a spring biased pressure valve may be formed in the tip of the applicator such that pressure exerted by the plunger overcomes the bias of the spring to open the valve and release the liquid.

In a preferred embodiment, the liquid is contained in a thin walled packet that is easily ruptured. The packet is placed inside the sheath before the plunger is inserted and before the applicator is packaged for dispensing to the consumer. When the consumer is ready to expel the liquid, the consumer presses the plunger, rupturing the packet and expelling the liquid through the hole in the tip of the applicator. In this embodiment, no rubber head is required on the plunger. Resistive force encountered by the plunger is provided by a detent in the sheath or the packet wall until the packet ruptures. Once the wall ruptures, the liquid can easily exit the cavity formed between the head of the plunger and an interior surface of the tip of the sheath through the relatively large hole and no substantial pressure in the liquid is reached which might force a significant amount of liquid past the head of the plunger. Further, any liquid which passes the head of the plunger is still retained within the sheath and causes no problems for the consumer.

The applicator just described comprising a sheath, a plunger, and a thin walled packet can be inexpensively manufactured so that it is economical to dispose of after a single use. The sheath and the plunger can be made of thermoplastic such as styrene in a standard two-part mold with a single insert for the center of the sheath. If a vapor or gas barrier is required for the wall of the packet, it can be made of aluminum foil or aluminum foil coated with a thin layer of easily ruptured thermoplastic such as polyethylene or styrene. The use of single dose packets minimizes waste of the treatment liquid. Because the treatment liquid is typically much more expensive than the described disposable applicator, the production cost per delivered dose using this embodiment of the present invention is lower than the production cost per dose using other applicator systems.

In another aspect, the invention is a method for delivering a dose of liquid at a spot using an applicator comprising a sheath with a tip at the hole, a packet of liquid inside the sheath, and a plunger, placing the tip of the sheath along side a spot where liquid is to be delivered, and pressing the plunger into the sheath thereby rupturing the packet and forcing liquid from the packet out the hole onto the spot.

In yet another aspect, the invention is a novel applicator comprising a sheath and a plunger which form a syringe where the syringe has a hole in the side of the tip leading to an outer surface of the tip, which is generally at an angle with the applicator between zero and 45 degrees, where the surface surrounding the hole has low curvature. This approximately flat surface is effective for pressing the liquid expressed through the hole against tissue such as a sore in the mouth. If the liquid is a film forming gel such as a cyanoacrylate based product, the approximately flat surface will hold the liquid against the sore while it begins curing to form a film.

### BRIEF DESCRIPTION OF THE DRAWINGS

The features of the present invention which are believed to be novel are set forth with particularity in the appended claims. Aspects of the invention may best be understood by making reference to the detailed description below in conjunction with the drawings wherein:

FIG. 1 shows a side view cross-section of an embodiment of the sheath and plunger.

FIG. 2 shows an end view cross-section of the sheath at the location of the hole.

FIG. 3a shows a top view cross-section of an embodiment of the sheath and plunger.

FIGS. 3b through 3f show cross-sections of one embodiment of the head of the plunger at cross section locations shown in FIG. 3a.

FIG. 4 shows a typical packet of liquid for use with the applicator.

### DETAILED DESCRIPTION

In the following detailed description of exemplary embodiments of the invention, reference is made to the accompanying drawings, which form a part hereof. The detailed description and the drawings illustrate specific exemplary embodiments by which the invention may be practiced. It is understood that other embodiments may be utilized, and other changes may be made, without departing from the spirit or scope of the present invention. The following detailed description is therefore not to be taken in a limiting sense, and the scope of the present invention is defined by the appended claims.

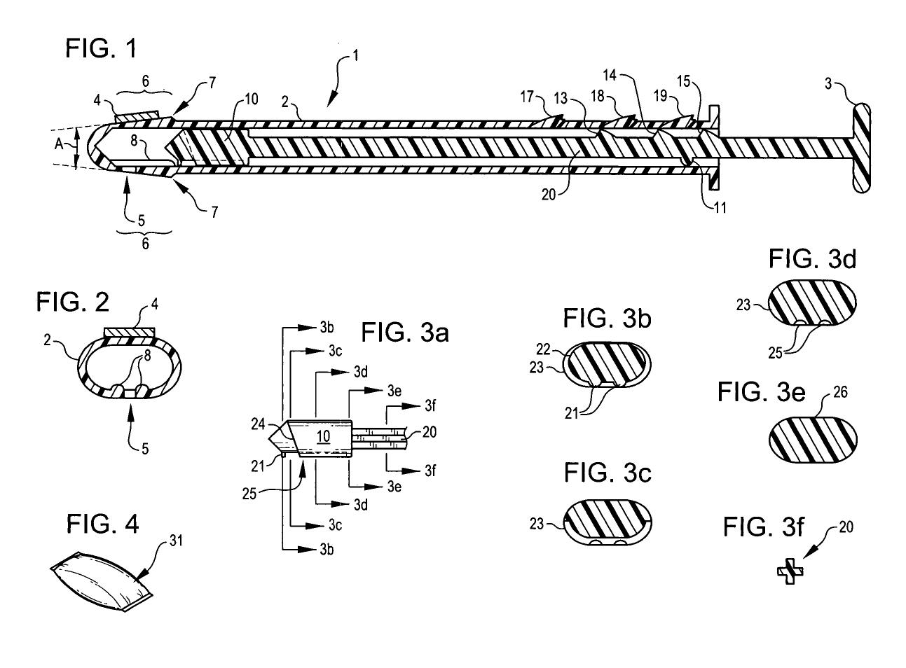

As shown in FIG. 1, an embodiment of the tactile applicator 1 comprises a sheath 2, a plunger 3, and an absorbent pad 4. The sheath and plunger are preferably made of injection molded plastic such as styrene or PVC or polypropylene. The head 10 of the plunger 3 fits snuggly inside the sheath 2, particularly at the tip of the sheath where the head of the plunger acts as a piston inside a cylinder. For adequate ability to reach into a mouth, the sheath has an interior length exceeding 90 millimeters. To make the applicator easy to handle, the interior has cross section with a maximum diameter less than 9 millimeters.

The sheath 2 includes a hole 5 near its tip. Each side of the tip of the applicator, both surrounding the hole 5 and under and surrounding the pad 4, is a slightly raised surface 6. The fact that it is raised relative to the sides of the sheath 2 creates a shoulder on each side 7. The raised surfaces may be inclined so that their high points are at the shoulders 7, narrowing to the tip to create an angle A between them. Each surface is at an angle with the passage in the sheath between 0 and 45 degrees. One raised surface forms a low curvature surface around the hole with less curvature than a radius of 4 millimeters, the surface extending at least 2 millimeters in every direction around the hole for spreading the liquid expressed from the hole.

A cross-section of the sheath at the location of the hole 5 is shown in FIG. 2. The pad 4, which is made of cellulose or cotton or other absorbent material, is glued to a side of the sheath around the hole or over the hole or, as shown in FIG. 2, opposite the hole 5. Along any of the four sides of the interior of the sheath 2, one or two detent strips 8 are added to prevent the plunger 3 from being pushed into the tip of the sheath prematurely. The head 10 of the plunger 3 rests against the detent strips 8 before the plunger is activated. As shown in one embodiment in FIG. 2, there may be two detent strips, one on each side of the hole 5. Placement of the strips in this location serves a second function of preventing the ruptured packed casing from blocking flow of liquid out the hole.

Not shown, a detent bump is formed in an inner surface of the sheath 2, by deforming the sheath with a punch from the outside after it is molded or by forming the sheath using a mold with inserts. The detent bump retains a shoulder formed on at least one side of the plunger 3.

In one embodiment, the applicator may not be adjustable in the amount of liquid dispensed except by adjustment at the factory of the amount of liquid placed in each packet. In an alternative embodiment, the applicator may include an adjustable stop that adjusts a maximum depth that the plunger extends into the sheath, thereby adjusting a cavity remaining in the tip when the head of the plunger reaches the maximum depth.

An embodiment of the adjustable stop is shown in FIG. 1. In this embodiment, the plunger includes several catchment ledges 13, 14, 15 molded into at least one side of the plunger 3. Each ledge can be caught by a moveable detent 17, 18, 19 molded into at least one side of the sheath. Each moveable detent is in the shape of a wedge where its point is adhered to plastic of the sheath by a thin hinge-like molded plastic structure. The other three sides of each moveable detent are separated from the surrounding plastic of the sheath by the outer mold which forms the sheath. The end of each moveable detent 17, 18, 19 opposite the hinge includes a ledge which snaps past the wall of the sheath and then catches on the wall of the sheath when the moveable detent is pressed by fingers of a user. In one embodiment, the first moveable detent is designated “−1”, the second moveable detent is designated “−2”, and the third moveable detent is designated “−3”. When the first detent is engaged, the plunger is stopped just short of its full stroke. When the second is engaged (whether or not the first is also engaged), the plunger is stopped a bit shorter of its full stroke. When the third moveable detent is engaged (whether or not any or both of the other two detents are engaged), the plunger is stopped still further from the full stroke.

Alternatively, instead of moveable detents, the adjustable stop may be formed with a threaded nut on the plunger which engages threads on the side of the plunger. Or, it may be formed by protrusions on the side of the plunger which can be bent by the fingers or broken to move out of blocking the plunger, allowing it to pass further into the sheath.

As a further alternative design for the adjustable stop which yields a two-way choice between the full dose or a reduced dose amount, the head of the plunger 3 and the interior of the tip of the sheath 2 may be asymmetrical. The points of each structure are offset to one side so that if the plunger is inserted in one of the two possible ways of inserting it, the offsets mesh and the plunger entirely fills the end of the tip of the sheath. However, if the plunger is removed, rotated 180 degrees, and reinserted, the offsets are not aligned and a void space cavity is left when the head 10 of the plunger 3 rests against the interior surface of the tip of the sheath 2.

FIG. 3a shows a side view of the head 10 of the plunger 3 and marks five cross sections of the head of the plunger and the shaft of the plunger.

The molding of the head 10 of the plunger 3 leaves a sharp point at the end. After the sharp point, the head flares in all directions until it reaches a shoulder at the location of cross-section 3b. From this shoulder until a ledge 24 is reached, the head of the plunger is does not fill the entire cross section of the sheath, leaving a slim gap through which liquid can pass as shown in FIG. 3b which shows the circumference of the narrowed tip 22 at the shoulder and shows two retaining nibs 21. In the cross section view, the circumference 23 of the head 10 surrounds the narrowed tip 22 of the head. As shown in FIG. 3a, the retaining nibs 21 are molded to be thin and easily deformable. They press against the detent strips 8 before the plunger is pressed. When the plunger is pressed, the detent strips 8 deform the retaining nibs 21.

FIG. 3c shows the cross-section at 3c. The inner ovoid line of FIG. 3c shows the perimeter of the head at this cross section and the outer line shows the circumference 23 of the head behind the cross-section. The space between the inner line and the outer line is small but large enough to allow a flow of fluid from the ruptured packet. The rear of this fluid flow space is defined by the diagonal ledge 24 which also shows in FIG. 3a. At cross-section 3b, the fluid flow space surrounds the head. At cross-section 3c, the fluid flow space extends around half of the head and includes clearance for the detent strips 8, as shown in FIG. 3c.

FIG. 3d shows the cross-section at 3d which is rearward of the diagonal shoulder 24. Grooves 25 in the circumference 23 snuggly accommodate the detent strips 8.

FIG. 3e shows the cross-section at 3e which is at the rear most portion of the head 10. At this cross-section, the circumference 26 includes no grooves for the detent strips 8 because the detent strips are not long enough to pass under this part of the head when the plunger is fully applied.

FIG. 3f shows the cross-section at 3f of the plunger shaft 20. The shaft is designed to be strong and easily molded. To reduce friction, most parts of the plunger shaft are narrow enough to not touch the sides of the sheath most of the time. As shown in FIG. 1, the catchment ledges 13, 14, 15 provide a gliding surface for guiding the plunger on one side and a gliding bump 11 is formed on an opposite side for guiding the plunger. Gliding bumps similar to gliding bump 11 are formed on the remaining two sides of the shaft 20 that are not shown in FIG. 1.

FIG. 4 shows a typical packet 31, in the shape of a pillow, containing liquid to be applied by the applicator. The packet is made of thin walled, low strength film or foil, such as aluminum or thin polyethylene or styrene or a combination of both, so that it will easily be ruptured by the plunger 3 when the plunger is pushed into the sheath. To facilitate rupture, the head 10 of the plunger 3 includes a sharp point of the same molded thermoplastic as the plunger.

Although the present invention has been described in considerable detail with reference to certain preferred embodiments, other embodiments are possible. Therefore, the spirit or scope of the appended claims should not be limited to the description of the embodiments contained herein. It is intended that the invention resides in the claims hereinafter appended as they may be amended.

1. A method for self-directing by touch a dose of liquid to be applied at a spot, comprising:
a. having an applicator comprising a sheath having a tip with a hole and, associated with the hole, a tactile surface, the applicator containing a liquid inside the applicator;
b. by feeling the tactile surface contacting touch sensitive tissues, placing the tip of the applicator along side a spot where liquid is to be delivered; and
c. activating the applicator, thereby releasing liquid from the hole onto the spot.

2. The method of claim 1 wherein the tactile surface surrounds the hole.

3. The method of claim 1 wherein the tactile surface is beside the hole.

4. The method of claim 1 wherein the tactile surface is on an opposite side of the tip from the hole.

5. The method of claim 1 wherein the tactile surface is absorbent.

6. The method of claim 1 wherein the liquid is enclosed by a thin-walled packet.

7. The method of claim 6 wherein the packet is made of thermoplastic.

8. The method of claim 1 wherein the liquid is delivered in a mouth for treating a mouth ulcer.

9. The method of claim 8 wherein the liquid is a film forming gel.

10. An applicator with a tactile surface, comprising:
(a) a sheath having an interior cavity and a tip with a hole communicating with the cavity and,
(b) associated with the hole, a tactile surface.

11. The applicator of claim 10 wherein the tactile surface surrounds the hole.

12. The applicator of claim 10 wherein the tactile surface is beside the hole.

13. The applicator of claim 10 wherein the tactile surface is on an opposite side of the tip from the hole.

14. The applicator of claim 10 wherein the tactile surface is absorbent.

15. The applicator of claim 10 further comprising an adjustable stop that adjusts a maximum depth that a plunger extends into the sheath, thereby adjusting a cavity remaining in the tip when the head of the plunger reaches the maximum depth.

16. The applicator of claim 15 wherein the stop comprises a set of movable detents affixed to at least one side of the sheath, each detent being movable from a starting position that does not restrain movement of the plunger to a set position that limits movement of the plunger in the sheath.

17. The applicator of claim 16 wherein the detents are each molded into the at least one side of the sheath as an integral molded unit with the sheath.

18. The applicator of claim 10 wherein the sheath is non-round in cross section.

19. The applicator of claim 18 wherein the sheath has an ovoid exterior cross section.

20. A method for delivering a dose of liquid at a spot, comprising:
a. having an applicator comprising a sheath having a tip with a hole and a plunger having a head, the applicator containing a packet of liquid in a cavity defined by an interior surface of the tip and the head of the plunger;
b. placing the tip of the sheath along side a spot where liquid is to be delivered; and
c. pressing the plunger into the sheath, thereby rupturing the packet and forcing liquid from the packet out the hole onto the spot.

21. The method of claim 20 wherein the packet is made of thermoplastic.

22. The method of claim 20 wherein the liquid is delivered in a mouth for treating a mouth ulcer.

23. The method of claim 22 wherein the liquid is a film forming gel.

24. An applicator with a rupturable packet of liquid, comprising a sheath having a tip with a hole and a plunger having a head, the applicator containing a packet of liquid in a cavity defined by an interior surface of the tip and the head of the plunger.

25. The applicator of claim 24 wherein the packet is made of thermoplastic.

26. The applicator of claim 24 further comprising an adjustable stop that adjusts a maximum depth that the plunger extends into the sheath, thereby adjusting a cavity remaining in the tip when the head of the plunger reaches the maximum depth.

27. The applicator of claim 26 wherein the stop comprises a set of movable detents affixed to at least one side of the sheath, each detent being movable from a starting position that does not restrain movement of the plunger to a set position that limits movement of the plunger in the sheath.

28. The applicator of claim 27 wherein the detents are each molded into the at least one side of the sheath as an integral molded unit with the sheath.

29. The applicator of claim 24 wherein the passage and plunger head are non-round in cross section.

30. An applicator, comprising:
a. a sheath having a tip, an open end opposite the tip, and a straight passage having a length exceeding 90 millimeters and a substantially constant cross section with a maximum diameter less than 9 millimeters connecting the open end with a hole through the sheath in a side of the tip, the hole leading to an orifice in a surrounding outer surface of the tip, the surface extending at least 2 millimeters in every direction around the orifice and, within said 2 millimeters, having less curvature than a radius of 4 millimeters, the surface generally at an angle with the passage between 0 and 45 degrees; and
b. a plunger at least as long as the passage, the plunger having a head that fits though the passage from the open end to the tip of the sheath beside the hole.

31. The applicator of claim 30 further comprising an adjustable stop that adjusts a maximum depth that the plunger extends into the sheath, thereby adjusting a cavity remaining in the tip when the head of the plunger reaches the maximum depth.

32. The applicator of claim 31 wherein the adjustable stop comprises a threaded nut surrounding the plunger and engaging threads on the plunger.

33. The applicator of claim 31 wherein the adjustable stop comprises a set of protrusions from a side of the plunger, each protrusion weakly adhered to the plunger so that it may be easily displaced with fingers to allow the plunger to pass into the sheath where the protrusion is attached.

34. The applicator of claim 31 wherein the stop comprises a set of movable detents affixed to at least one side of the sheath, each detent being movable from a starting position that does not restrain movement of the plunger to a set position that limits movement of the plunger in the sheath.

35. The applicator of claim 34 wherein the detents are each molded into the at least one side of the sheath as an integral molded unit with the sheath.

36. The applicator of claim 36 wherein the passage and plunger head are non-round in cross section.

37. The applicator of claim 36 wherein the passage and plunger head are ovoid in cross section.

38. The applicator of claim 30 further comprising a tactile surface on the tip associated with the hole.

39. The applicator of claim 38 wherein the tactile surface is an absorbent pad.
